(19) United States
(12) Patent Application Publication
(10) Pub. No.: US 2005/0071015 A1
(43) Pub. Date: Mar. 31, 2005

(54) Acetabular prosthesis assembly

(76) Inventor: Ronald Sekel, Matraville (AU)

Correspondence Address: Thomas M Galgano;Galgano & Burke, Suite 135, 300 Rabro Drive, Hauppauge, NY, 11788, US

(21) Appl. No.: 10/475,948

(22) PCT Filed: Apr. 26, 2002
(86) PCT No.: PCT/AU02/00509

(30) Foreign Application Priority Data
Apr. 26, 2001 (AU) PR 4579

(51) Int. Cl. A61F 2/34

(52) U.S. Cl. 623/22.28

(57) ABSTRACT

An acetabular prosthesis assembly (1) comprising an acetabular cup (3) with a generally convex outer surface for engaging acetabular bone and a generally concave inner surface, an insert (10) capable of insertion in the cup (3) for receiving a femoral head component, wherein a wear liner (13) is disposed between the cup (3) and the insert (10), the liner (13) providing a wear inhibiting surface (4a).

### BACKGROUND

The present invention relates to acetabular components and more particularly relates to an acetabular prosthesis assembly for implantation within an acetabulum and which includes a cup for fixation to bone, an insert for insertion in the cup and a liner which provides a wear surface opposing a concave surface of the in the cup.

### PRIOR ART

There are a wide variety of known acetabular components which restore and substitute for an internally damaged acetabulum. These are for local corrections such as in a total hip replacement surgery where the pelvis other than the acetabulum is in a good state of repair. Examples of known Acetabular cup arrangements incorporated herein by reference may be seen in U.S. Pat. Nos. 5,871,548, 5,782,929, 5,326,368, 5,480,448, 5,370702, 5,019,108. Some of the current art is directed to various aspects of Acetabular cup fixation design and to the manner of engagement between an insert and acetabular cup.

For example, U.S. Pat. No. 5,871,548 discloses an acetabular reinforcement system including a substantially cup shaped reinforcement body having a peripheral flange portion. The system includes one or more fixation wings of various sizes and shapes which are separately attachable to the flange portion of the reinforcement body. The system is mountable within the acetabulum of a patient to reinforce the acetabulum and to serve as a platform for other prosthesis components such as an acetabular shell.

An alternative acetabular arrangement is disclosed in U.S. Pat. No. 5,658,347. This patent discloses another cup for reconstruction of an acetabulum in a pelvis and a method for installing the apparatus. The cup is generally ellipsoidal in shape with a rigid keel extending from the convex surface of the cup at a given angle. An ellipsoidal concave cavity is formed in the pelvis and a stabilisation channel is formed into the pelvis at an angle which is close to but slightly less than the angle on the keel. The cup is installed in the concave cavity and the rigid keel is forced into the stabilisation channel. This keys in the acetabular cup.

The known acetabular assemblies generally comprise a metallic outer shell which forms the acetabular cup. The cup is preferably made from chrome cobalt or titanium and includes a generally convex outer surface and a concave inner surface. The outer surface is usually porous or roughened to promote bone growth thereabout. The cup receives a polyethylene, metal or ceramic insert which opposes a convex inner surface of the cup. The insert typically includes a recess which receives a femoral head component. The acetabular cup may alternatively receive a metal or ceramic insert.

In a typical acetabular assembly there are two main wear interfaces, the first between a convex outer surface of a cup insert and a concave inner surface of the cup (also referred to as a shell) and the second between a head of a femoral prosthesis and a concave recess in the insert. Polyethylene wear is a known problem in acetabular assemblies and can lead to conditions such as polyethylene disease involving serious bone degradation. This can occur where micro particles of polyethylene which exfoliate due to wear migrate to bone. In a typical acetabular prosthesis, this may occur through micro wear at the interface between the outer convex surface of a polyethylene insert and an inner convex surface of the cup which the insert engages. These particles can migrate to bone in the acetabulum. The micro wear particles can migrate through openings on an acetabular cup which are included for additional bone fixation.

Considerable work has been performed on providing a durable polyethylene with reduced wear characteristics but no polyethylene exists which is fire from wear. Highly crosslinked polyethylenes have been developed which significantly decrease (but do not eliminate) wear. Wear occurs between chrome cobalt and polyethylene and ceramic and polyethylene interfaces. Some work bas been done on ceramic on ceramic and metal on metal assemblies to reduce wear, particle generation and to provide more resistance to the adverse effects of minute particles. The problem of polyethylene wear has not been solved due to the nature of the material itself.

At present, acetabular cups (or shells) are provided of various diameters which will receive and retain metal, ceramic and polyethylene inserts. Thus a surgeon has a choice of insert which may be used on a particular patient but that choice does not overcome the problem of back surface polyethylene wear when a polyethylene insert is selected. As cup sizes typically range between 40 mm and 72 mm, large inventories are required. Different cup designs are required for polyethylene inserts, metal and ceramic liners. It would be desirable if inventories could be reduced by ensuring compatibility between acetabular cups and available inserts in all biomaterials namely metal, ceramic and polyethylene.

Although previous attempts have been made to address polyethylene micro wear problems, none have provided a satisfactory solution to date. One attempt to address the problem was to make the polyethylene insert which is traditionally separate from the outer acetabular cup, integral (moulded) with the acetabular cup to eliminate relative movement between the insert and the cup i.e. the outer shell. It was thought that by eliminating the potential for relative movement between the insert and the cup that polyethylene micro wear could be eliminated. The problem in this approach to solving the problem was that the surgeon lost the advantage of access to holes in the outer cup for additional bone fixation by screws. It also required that the geometry of the cup be perfect for fitting purposes as the surgeon did not have any further means of fixation once the cup was fitted in the case for instance of a sloppy or uneven engagement with the acetabulum. It is preferable that the cup when inserted be rotatably adjustable if realignment of the cup is required for fitting and fixation purposes. Another solution attempted was to eliminate the fixation holes on the outer cup to prevent passage to acetabular bone of any migrating polyethylene wear particles.

### INVENTION

The present invention is particularly concerned with improvements in the acetabular assemblies which employ polyethylene inserts and more particularly to the reduction of wear at the insert/cup interface and the elimination of the wear problems associated with the use of polyethylene inserts.

The present invention seeks to ameliorate the shortcomings of the prior art by providing an acetabular assembly of the type including a cup and polyethylene insert in which the assembly includes a liner of preferably metallic material which provides a wear surface between the insert and a concave inner surface of the acetabular cup. Preferably, the liner will be disposed on a rear convex surface of the polyethylene and is of like material to the acetabular cup.

In its broadest form the present invention comprises:

an acetabular prosthesis assembly comprising an acetabular cup having a generally convex outer surface for engaging acetabular bone and a generally concave inner surface together defining a cup wall which has an apex and an equator;
an insert capable of insertion inside the acetabular cup;
the insert including a convex outer surface and a concave recess which receives a femoral head component; wherein, the assembly further includes a liner disposed between the concave inner surface of the cup and the convex outer surface of the insert;
the liner providing wear inhibiting surface.
   Preferably the liner is a metallic material which opposes at least part of a concave inner surface of the cup and is integral with the inset.
   According to one embodiment, the insert comprises a first layer of polyethylene fused to the metallic liner which is preferably chrome cobalt or titanium alloy and which engage at least part of the inner concave surface of the cup.

The circumferential wall of the cup is increased in length a predetermined circumferential distance beyond the equator and this length. This distance will vary but will preferably fall within the range of 2 mm-5 mm. The thickness of the liner at its apex influences the circumferential distance that the cup extends from its equator. The liner is in tight fitting engagement with part of the inner concave surface of the cup leaving a gap between an outer surface of the liner and the inner surface of the cup.

The insert has a generally spherical outer surface opposing a generally spherical concave surface of the liner and the outer surface of the metallic liner includes a straight wall region extending from near its equator in the direction of the apex and an arcuate portion in the region of its apex, the arcuate portion opposing the inner concave surface of the cup. The insert includes a profile part which keys into a back surface of the metallic liner. Preferably, the cup has a substantially spherical outer surface and includes a flattened region at its apex.

The axial distance from the apex of the acetabular cup to its equator is preferably less that the radial distance from the circumferential wall at the equator to its axis.

The straight wall region of the liner is disposed at an angle within the range of 15-25 degrees, but not excluding other angles, from its axis of symmetry and changes to the arcuate portion at a location less than half the circumferential distance from its equator to its apex.

In another broad form the present invention comprises:

an insert for an acetabular cup; the insert including a convex outer surface and a concave inner surface defining a body including a recess which receives a femoral head component; wherein the convex outer surface of the insert body receives thereon a liner which provides a wear inhibiting surface opposing at least part of an inner surface of the cup which receives and, retains said insert.
   Preferably, the liner is formed from a metallic material which opposes at least part of a concave inner surface of the cup and is integral with the insert
   The insert body is polyethylene and the metallic liner is fused to the metallic liner which is titanium alloy or chrome cobalt and when the insert is inserted by the cup, engages at least part of an inner surface of the acetabular cup. The liner is in tight fitting engagement with part of the inner concave surface of the cup. A gap is left between an outer surface of the liner and an opposing inner surface of the cup to prevent the insert engaging the convex inner surface of the cup before the wall of the liner is fully and tightly engaged in its desired position. This prevents unwanted popping out of the prosthesis. The insert includes a profile part which keys into a back surface of the metallic liner wherein, the straight wall region of the liner is disposed at an angle usually within, but not exclusively, within the range of 15-25 degrees from its axis of symmetry. The straight wall region of the liner changes to said arcuate portion at a location less than half the circumferential distance from its equator to its apex.
   In another broad form the present invention comprises:
An acetabular cup assembly for repair of an acetabulum; the assembly comprising an acetabular cup generally defining a hemisphere having an exterior convex surface and an interior at least partially concave surface, an apex and an equator;
an insert for engagement with said cup and which receives a femoral head;
a liner between said insert and said cup providing a wear inhibiting surface;
wherein a wall of the acetabular cup extends a predetermined circumferential length distance beyond its equator away from its apex.
   Preferably, the predetermined distance the wall extends beyond the equator is related to the thickness of the liner which is interposed between the insert and said interior surface of said cup.
   The equatorial extension distance may be adjusted to accommodate liners of different thicknesses.
   In another broad form of the method aspect the present invention comprises: a method of construction of an acetabular cup assembly comprising the steps of;
a) forming an acetabular cup having a generally convex outer surface for engaging acetabular bone and a generally concave inner surface together defining a cup wall which has an apex and an equator,
b) taking an insert capable of insertion inside the acetabular cup; the insert including a convex outer surface and a concave recess which receives a femoral head c) applying a wear inhibit liner to the convex outer surface of the insert so that when the insert is inserted in the acetabular cup the liner is disposed between the generally concave inner surface of the cup and the convex outer surface of the insert.
   Preferably the method comprises the further step of increasing in length the circumferential wall of the cup a predetermined circumferential distance beyond the equator.
   The method also comprises the step of fusing the insert to the liver so that the liner is integral with the insert, whereupon the insert is inserted into the acetabular cup so that at least part of an outer wall of the liner engages at least part of the inner wall of the cup and the remainder of the outer wall of the liner defines a gap or space between the liner and the cup.
   The liner is provided with the outer surface of the metallic liner includes a straight wall region extending from near its equator in the direction of the apex and an arcuate portion in the region of its apex, the arcuate portion opposing the inner concave surface of the cup. The cup is provided with a substantially spherical outer surface and is formed so that an axial distance from the apex of the outer surface of the acetabular cup to its equator is less that the radial distance from the outer surface of the circumferential wall at the equator to its axis of symmetry.
   In another broad form the present invention comprises: an acetabular assembly comprising an acetabular cup a convex outer surface for engaging acetabular bone and a generally concave inner surface;
an insert for insertion inside the cup and which includes a concave recess which receives a femoral head component; characterised in that the insert further includes a layer or liner which opposes said generally concave inner surface of said cup and which provides a wear surface in the event of relative micro movement between said liner and said cup. Preferably, the insert comprises polyethylene with a metallic liner comprising a convex outer surface of the insert. The inner surface of the metallic liner may have an irregular porous surface to bind the polyethylene.
   In the two layer insert the chrome cobalt or titanium alloy liner may be abbreviated short of the extremity of the inner surface of the outer shell in which case the polyethylene liner may be increased in thickness at the abbreviation.
   In another broad form the present invention comprises; an acetabular cup for use with an acetabular assembly for repair of an acetabulum; the assembly comprising an acetabular cup and an insert for engagement with said cup and which receives a femoral head; the acetabular cup generally defining a hemisphere having an exterior convex surface and an interior concave surface, an apex and an equator, characterized in that a wall of the acetabular cup extends a predetermined circumferential distance beyond its equator. Preferably, the predetermined distance the wall extends beyond the equator is determined by the thickness of a liner which is interposed between a polyethylene insert and said interior concave surface of said cup.
   In another broad form the present invention comprises; an acetabular cup for use with an acetabular assembly for repair of an acetabulum; the assembly comprising an acetabular cup and an insert for engagement with said cup and which receives a femoral head; the acetabular cup generally defining a hemisphere having an exterior convex surface and an interior concave surface an apex and an equator; characterized in that the wall of the acetabular cup extends a predetermined circumferential distance beyond its equator, wherein said wall extension is effected to accommodate an insert including a liner which provides a wear resistant surface. The equatorial extension distance may be adjusted to accommodate liners of different sizes. In the case of thinner liners the extension distance will be smaller than the extension required for thicker liners.

### DETAILED DESCRIPTION

The present invention will now be described in more detail according to a preferred but non limiting embodiment and with reference to the accompanying illustrations; wherein

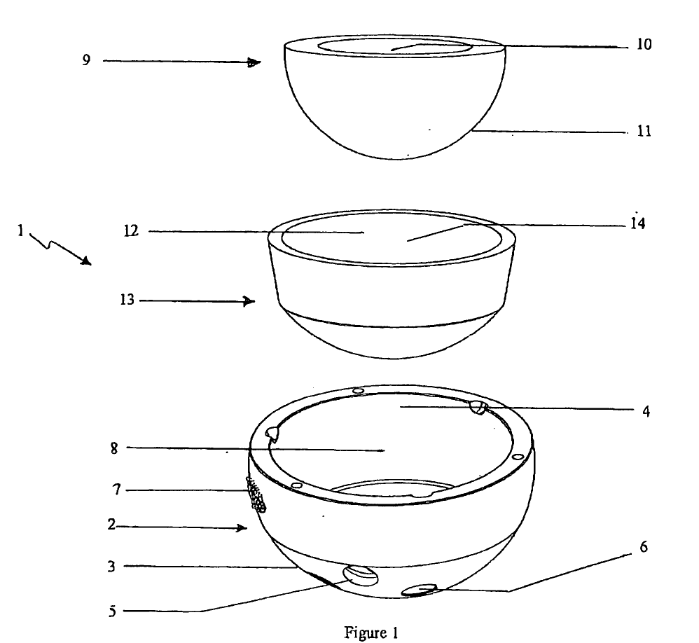

FIG. 1 shows an exploded view of an acetabular cup assembly according to a preferred embodiment

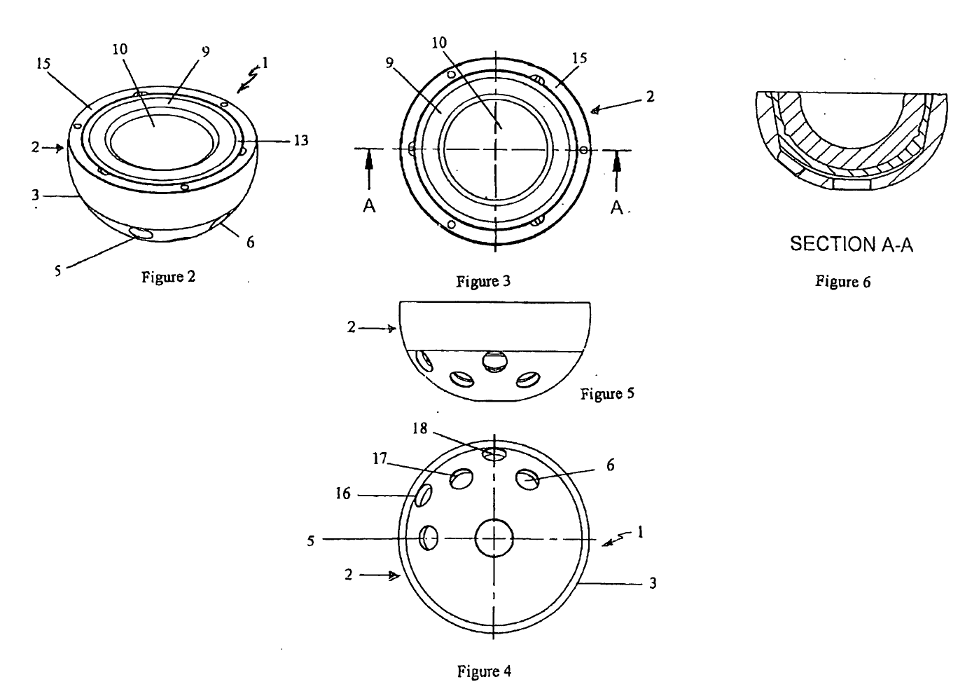

FIG. 2 shows a perspective view of the assembly fully assembled.

FIG. 3 shows a top plan view of the assembly of FIG. 2.

FIG. 4 shows a bottom plan view of FIG. 2 showing the outer surface of the cup.

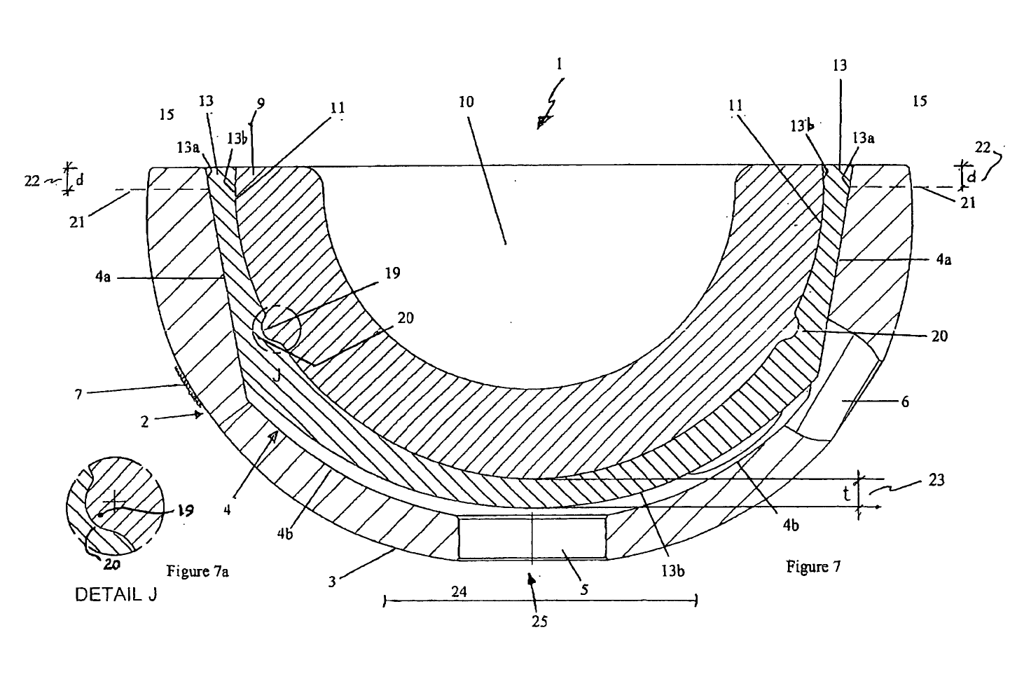

FIG. 5 shows a side elevation of the assembly of FIG. 2.

FIG. 6 shows a cross section through the assembly along line AA shown in FIG. 3

FIG. 7 shows an enlarged cross sectional view of an acetabular assembly geometry according to a preferred embodiment of the invention.

FIG. 7a shows an enlargement of a male female key for engagement between the liner and insert

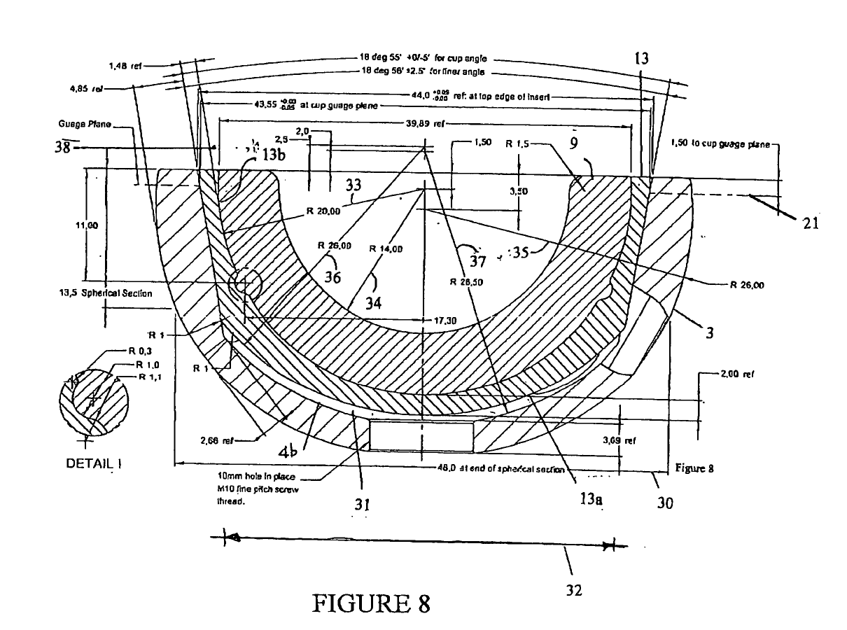

FIG. 8 shows the enlarged cross sectional view of FIG. 7 including proportionality parameters of a preferred but non limiting embodiment.

Referring to FIG. 1 there is shown an exploded view of an acetabular assembly 1 according to a preferred embodiment of the invention. Assembly 1 comprises standard hemispherical acetabular cup 2 including a generally arcuate body having a generally convex outer surface 3 which would normally vary in radii between 44 mm and 72 mm and a generally concave liner surface 4. Cup 2 typically includes apertures 5 and 6 which is receive fixation screws (not shown) for fixation to acetabular bone. Convex surface 3 includes a porous coating 7 to promote bone growth about the cup. Internal space 8 receives and retains an insert 9 which includes recess 10 for receiving a femoral head component (not shown). According to the prior art this insert would typically be made from metal, ceramic or polyethylene. Insert 9 includes an internal concave recess 10 having radii which may typically fall between 22 and 32 mm.

Insert 9 includes a convex outer surface 11 which engages a corresponding inner generally concave surface 12 of liner 13. Liner 13 includes a recess 14 which engages surface 11 of insert 9.

Known acetabular assemblies include full thickness chrome cobalt or ceramic inserts which oppose the concave inner surface of an acetabular cup. The concave inner surface of the cup has a tapered profile which receives opposing mating tapered surfaces. According to the embodiment of FIG. 1, assembly 1 comprises an insert formed from polyethylene and engages a liner 13 which is preferably formed from chrome cobalt or titanium alloy. The polyethylene may be injection moulded onto the metallic liner whereupon liner 13 will seal outer surface 11 of polyethylene insert 9.

FIG. 2 shows a top perspective view of the assembly of FIG. 1 fully assembled. In this view, it can be seen that liner 13 which has been fused to insert 9 fits neatly within wall 15 of cup 2. A femoral head component (not shown) fits within recess 10.

FIG. 3 shows a top plan view of the assembly of FIG. 2 and bears corresponding numbering for corresponding parts. FIG. 4 shows a bottom plan view of FIG. 2 showing the outer convex surface 3 of the cup 2 with fixation openings 5, 6 16, 17 and 18. The openings may be distributed in other locations than those shown about cup 2 according to requirements.

FIG. 5 shows a side elevation of the assembly of FIG. 2 with fixation openings. FIG. 6 shows a cross section through the assembly along line AA shown in FIG. 3. FIG. 6 shows an enlarged cross sectional view of the acetabular assembly of FIG. 1 according to a preferred embodiment of the invention.

Acetabular assembly 1 according to a preferred embodiment of the invention comprises standard generally hemispherical acetabular cup 2 including a generally arcuate body having a generally convex outer surface 3 which would normally vary in radii between 44 mm and 72 mm and a generally concave inner surface 4. Concave inner surface 4 is formed to accommodate at least part of an outer contour of liner 13. Concave inner surface 4 comprises planar region 4a and an arcuate portion 4b. Tight fitting engagement between cup 2 and liner 13 is via opposing surfaces 4a and 13a It is preferred that liner 13 does not engage inner concave surface 4b of cup 2 to ensure that the liner 13 does not engage surface 4b prior to complete engagement between opposing surfaces 4a and 13a. Should surface 13b of liner 13 engage surface 4b before insert 9 with fused liner 13 is fully seated within cup 2 and before surfaces 13a and 4a are fully engaged, insert 9 and liner 13 could spring out of the cup. Cup 2 typically includes apertures 5 and 6 which receive fixation screws (not shown) for additional fixation of acetabular cup 2 to acetabular bone. Convex surface 3 includes a porous coating 7 to promote acetabular bone growth about the cup enhancing fixation. Liner 13 further comprises surface 13c which opposes convex surface 11 of insert 9. Insert 9 includes recess 10 for receiving a femoral head component (not shown). Recess 10 has radii measured from its axis of symmetry to its circumference which may typically fall between 22 and 32 mm. Insert 9 is fused to liner 13 via surfaces 13 b and 11. Engagement between these surfaces is enhanced using a male profile part 19 in insert 9 which engages female recess 20 in liner 13 (see enlargement FIG. 7a) surface includes a convex outer surface 11 which receives a corresponding inner generally concave surface 12 of liner 13. Liner 13 defines a recess 14 (see FIG. 1) which receives surface 11 of insert 9.

According to one embodiment, liner 13 may be abbreviated short of the extremity of the inner surface 4a of the cup 2 in which case the polyethylene insert 9 may be increased in thickness at the abbreviation. The liner 13 preferably will be finished as a taper with the same profile as the internal sure 4a of the cup 2. The polyethylene insert 9 is sealed to the chrome cobalt (or titanium) metal layer or by pressure or injection moulding. This eliminates wear on the polyethylene convex outer surface 11 which would otherwise be the case and allows metal to metal contact between surface 13a of liner 13 and inner surface 4a of cup 2. Thus, by use of the embodiment described, any micro wear is confined to metal on metallic thereby eliminating wear induced polyethylene micro particles. Any micro movement between the insert and cup will therefore result in metallic rather than polyethylene wear. Metallic wear is preferred to polyethylene wear as metallic wear particles are non toxic.

As may be seen from FIG. 7, wall 15 of cup 2 passes beyond equator 21 a predetermined distance d represented by numeral 22. Distance 22 is related to the thickness t which is represented by numeral 23 of liner 13. This extension beyond equator 21 by distance d will normally be within the range 2-5 mm but ideally will be close to or at 3 mm.

Accordingly, the wall of a typical cup applied in accordance with the invention will normally extend beyond equator 21 to accommodate the extra thickness of liner 13. The extension beyond equator 21 enables insertion of liner 13 which takes up additional space in the cavity formed inside cup 2. In the absence of an extension to cup 2 at equator 21, insert 9 would due to the presence of liner 13 protrude beyond cup wall 15 if wall 15 terminated at the cup equator. Insert 9 and liner 13 must be fully retained in the cup 2 to ensure the required degree of tight interfitting.

When an acetabular assembly of the type described is to be inserted the surgeon prepares the acetabular cavity by reaming with a spherical reamer. The spherical reamer will allow formation of a cavity in the acetabulum of predetermined depth and diameter which neatly accommodates outer convex surface 3 of cup 2. Although cup 2 is for the most part spherical, the cup includes a slightly flattened apex region 24. This flattening or abbreviation is introduced to ensure the cup surface 3 does not preferentially bottom on the apex (not shown) of a reamed acetabular surface to which the assembly is to be fitted before the assembly is properly located in its predesignated position. In the absence of this geometry, the assembly may undergo unwanted withdrawal from the acetabular cavity. The objective is to ensure that the cup positively engages the circumferential wall of the reamed acetabulum. This ensures preferential press fit engagement between cup 2 and an acetabular side wall rather than prematurely with the acetabular bone apex.

In the case where a polyethylene insert is used, this will have a wall thickness of around 6 mm and may receive a femoral head having a diameter 26, 28 or 32 mm. The diametrical size of the head may normally increase according to the diameter of the selected cup. However, it has been found that a 6 mm polyethylene liner is the smallest wall thickness which can optimally accommodate applied loadings generated by the femoral head.

In the prior art, the dimensions (0, R1) where R1 is the distance from the equator to the apex of the cup and (R2, 0) where R2 is the radial length at the equator will measure approximately 24 mm. In a typical 50 mm cup with a polyethylene insert, there is according to the prior art arrangements no remaining space within the cup for any elements other than the insert. It is an objective of the present invention to provide an alternative to the known acetabular assemblies and which eliminates the problem of polyethylene wear which can lead to polyethylene bone disease. More particularly it is an objective to eliminate polyethylene wear caused by micro movement of the outer convex face of the polyethylene which opposes the inner concave surface of the cup. This is achieved by providing the metallic liner 13 which engages the outer surface of the polyethylene insert 11. The problem arises as to how to accommodate an additional thickness of the liner for a given diametrical size cup but still accommodate a metal, ceramic or polyethylene liner with one inventory of cups and with single internal geometry. As cups come in an inventory of standard sizes it would be undesirable to alter known sizes but an advantage to accommodate the invention within the standard and special order known diametric cup sizes. For a selected cup size, if a wear resistant liner is to be placed between a polypropylene insert and an acetabular cup the extra thickness of the liner will naturally displace the insert axially by at least the thickness of the insert. In smaller cup sizes, it is not advisable to overcome this change in geometry by reducing the thickness of the polypropylene below 6 mm, to preserve the existing cup equator termination point for the reasons indicated earlier as that would compromise wear characteristics in the polypropylene between the femoral bead and insert.

According to one embodiment, a preferred solution to the problem is to allow the insert to advance axially and to accommodate the advance by extending the equator of cup 2 circumferentially to approximately the distance of the thickness near the apex of the wear liner 13.

FIG. 8 shows the enlarged cross sectional view of FIG. 7 including proportionality parameters of a preferred but non limiting embodiment.

A 50 mm acetabular cup, will have an equatorial outside diameter of 52 mm to ensure press fit engagement with an acetabular wall. The distance from the equator 21 of the 50 mm cup to the apex 25 will be 25 mm and the radial equatorial distance will be 26 mm. Thus the radial distance will be slightly greater than the distance to the apex due to the flattened region 24 as previously described. The cup 2 will preferably be 34 mm thick and will include the porous outer coating 7. At the equator wall 15 of cup 2 includes an extension region 22 which extends from the equator and follows the circumference for a predetermined distance which in the case of a 50 mm cup will be about 2-3 mm to accommodate liner 13. Extension of the wall 15 beyond the equator 21 of cup 2 allows an optimum minimum thickness of polyethylene to be maintained.

Known acetabular cups form a hemisphere whose circumferential walls terminate at their equator. In order to introduce a wear resistant surface where polyethylene inserts are used with similar geometry to metal and ceramic liners, the cup wall must extend beyond its equator to accommodate the consequent axial displacement of the polyethylene insert due to the addition of the wear resistant metal surface which will ideally have a thickness in the order of 2-4 m.

As the extension 22 to wall 15 follows the circumference of cup 2 the extension will naturally have a curved outer surface with the opposing inner surface 4a profiled to match a corresponding surface 13a on liner 13.

One advantage of the invention will be that the fused multi layer polyethylene/metallic insert with liner can be interchanged with known metal and ceramic inserts which will keep inventories to a minimum. It will be possible to insert thicker inserts into smaller cups due to the equatorial extension in the wall of the cup. The wall of the cup will typically reach a predetermined distance beyond the equator. The distance of the extension may be designed according to the strength required at the extremity but it would be expected to be in the order of 2-4 mm. Another advantage of the assembly is that the surgeon keeps the advantage of access to additional fixation openings in the acetabular cup component and the plastics on metal wear interface of the prior art polyethylene/metal is eliminated.

FIG. 8 the enlarged cross sectional view of FIG. 7 including proportionality parameters of a preferred but non limiting embodiment. Numbering used to identity components of the assembly in FIG. 7 has been removed for clarity. A comparison of FIG. 8 with FIG. 7 will enable identification of corresponding parts. FIG. 8 demonstrates with reference to radial distances and thicknesses the proportions of a typical assembly according to one embodiment of the invention. Arrow 30 represents a distance where the sphericity in the cup is slightly flattened to ensure that the outer surface 3 of Cup 2 does not bottom out in a reamed acetabulum prior to full and proper location of the assembly in a patient. This flattened region may be abbreviated over a distance shown by numeral 24 in FIG. 7. A gap 31 is formed between surface 13a of liner 13 and surface 4b of cup 2. Gap 31 is enables by a local thinning in the wall of cup 2 over the approximate distance indicated by arrow 32. Radii of surfaces of the assembly components are indicated by Radial co ordinates in the figure. Radial lengths of insert 9 are taken at the equator 21. Outer radial distance of surface 13 b is indicated by arrow 33 defining R 20,00. Inner radial distance of surface 11b is indicated by arrow 34 defining R 14,00. Outer radial distance of surface 3 is indicated by arrow 35 defining R 20,00.

Outer radial distance of liner surface 13a surface 13 b is indicated by arrow 36 defining R 26,00. Outer radial distance of surface 4 b is indicated by arrow 37 defining R 26, 50. Angle of repose of the wall of liner 13 defined by boundaries 13 a and 13b of liner 13 is identified by numeral 38. FIG. 8 shown but one possible geometry for the assembly according to one embodiment. Other embodiments within the invention are possible and these may be altered according to prescribed criteria.

It will be recognized by persons skilled in the art that numerous variations and modifications may be made to the invention broadly described herein without departing from the overall spirit and scope of the invention.

1. An acetabular prosthesis assembly comprising an acetabular cup having a generally convex outer surface for engaging acetabular bone and a generally concave inner surface together defining a cup wall which has an apex and an equator; an insert capable of insertion inside the acetabular cup; the insert including a convex outer surface and a concave recess which receives a femoral head component; wherein, the assembly further includes a liner disposed between the concave inner surface of the cup and the convex outer surface of the insert; the liner providing wear inhibiting surface.

2. An acetabular assembly according to claim 1, wherein the liner is a metallic material which opposes at least part of a concave inner surface of the cup.

3. An acetabular assembly according to claim 2, wherein the liner is integral with the insert.

4. An acetabular assembly according to claim 3 wherein, the insert comprises a first layer of polyethylene fused to said metallic liner material.

5. An acetabular assembly according to claim 4, wherein the metallic liner is titanium alloy or chrome cobalt and engages at least part of the inner concave surface of the cup.

6. An acetabular assembly according to claim 6 wherein the circumferential wall of the cup is increased in length a predetermined circumferential distance beyond the equator.

7. An acetabular assembly according to claim 5 wherein the thickness of the liner at its apex influences the circumferential distance that the cup extends beyond its equator.

8. An acetabular assembly according to claim 7 wherein the liner is in tight fitting engagement with part of the inner concave surface of the cup.

9. An acetabular assembly according to claim 8 further comprising a gap between an outer surface of the liner and said inner surface of the cup to prevent unwanted engagement between the outer surface of the liner and said inner surface of the cup.

10. An acetabular assembly according to claim 9 wherein the circumferential wall of the cup is extended between [2 MM-5 MM] from the equator.

11. An acetabular assembly according to claim 10 wherein; the insert has a generally spherical outer surface opposing a generally spherical concave surface of the liner.

12. An acetabular assembly according to claim 11 wherein the outer surface of the metallic liner includes a straight wall region extending from near its equator in a direction of the apex and an arcuate portion in the region of its apex, the arcuate portion opposing the inner concave surface of the cup.

13. An acetabular assembly according to claim 12, wherein the cup has a substantially spherical outer surface.

14. An acetabular assembly according to claim 13 wherein the insert includes a profile part which keys into a back surface of the metallic liner.

15. An acetabular assembly according to claim 14 wherein, the cup includes a flattened region at its apex.

16. An acetabular assembly according to claim 15 wherein the axial distance from the apex of the acetabular cup to its equator is less that the radial distance from the circumferential wall at the equator to its axis.

17. An acetabular assembly according to claim 16 wherein, the straight wall region of the liner about is disposed at an angle within the range of 15-25 degrees from an axis of symmetry of the assembly.

18. An acetabular assembly according to claim 17 wherein, the straight wall region of the liner changes to said arcuate portion at a location less than half the circumferential distance from its equator to its apex.

19. An acetabular assembly according to claim 18 where the insert is made of polyethylene.

20. An acetabular assembly according to claim 19 wherein the liner is made of either chrome cobalt or titanium alloy.

21. An insert for an acetabular cup; the insert including a convex outer surface and a concave inner surface defining a body including a recess which receives a femoral head component; wherein the convex outer surface of the insert body receives thereon a liner which provides a wear inhibiting surface opposing at least part of an inner surface of the cup which receives and retains said insert.

22. An insert for an acetabular cup according to claim 21, wherein the liner is formed from a metallic material which opposes at least part of a concave inner surface of the cup.

23. An insert for an acetabular cup according to claim 22 wherein, the liner is integral with the insert.

24. An insert for an acetabular cup according to claim 23 wherein the insert body is polyethylene and the metallic liner is fused to said polyethylene.

25. An insert for an acetabular cup according to claim 24 wherein, the metallic liner is titanium alloy or chrome cobalt and when the insert is inserted by the cup, engages at least part of an inner surface of the acetabular cup.

26. An insert for an acetabular cup according to claim 25 wherein a circumferential wall of the cup is increased in length a predetermined circumferential distance beyond an equator of the cup.

27. An insert for an acetabular cup according to claim 26 wherein the thickness of the liner at its apex influences the circumferential distance that the wall of the cup is extended from the equator.

28. An insert for an acetabular cup according to claim 27 wherein the liner is in tight fitting engagement with part of the inner concave surface of the cup.

29. An insert for an acetabular cup according to claim 28 wherein, when the insert is inserted in the cup a gap is left between an outer surface of the liner and an opposing inner surface of the cup.

30. An insert for an acetabular cup according to claim 29 wherein the circumferential wall of the cup is extended in length between 2 mm-5 mm from the equator.

31. An insert for an acetabular cup according to claim 30 wherein; the insert has a generally spherical outer surface opposing a generally spherical concave surface of the liner.

32. An insert for an acetabular cup according to claim 31 wherein the outer surface of the metallic liner includes a straight wall region extending from near its equator in the direction of the apex and an arcuate portion in the region of its apex, the arcuate portion opposing the inner concave surface of the cup.

33. An insert for an acetabular cup according to claim 32 wherein the insert includes a profile part which keys into a back surface of the metallic liner.

34. An insert for an acetabular cup according to claim 33 wherein, the straight wall region of the liner is disposed at an angle within the range of 15-25 degrees from its axis of symmetry.

35. An insert for an acetabular cup according to claim 34 wherein, the straight wall region of the liner changes to said arcuate portion at a location less than half the circumferential distance from its equator to its apex.

36. An insert for an acetabular cup according to claim 35 wherein the insert is made of polyethylene.

37. An insert for an acetabular cup according to claim 36 wherein the liner is made of either chrome cobalt or titanium alloy.

38. An acetabular cup assembly for repair of an acetabulum; the assembly comprising an acetabular cup generally defining a hemisphere having an exterior convex surface and an interior at least partially concave surface, an apex and an equator; an insert for engagement with said cup and which receives a femoral head; a liner between said insert and said cup providing a wear inhibiting surface; wherein a wall of the acetabular cup extends a predetermined circumferential distance beyond its equator in a direction away from its apex.

39. An acetabular cup assembly according to claim 38 wherein the predetermined distance the wall extends beyond the equator is related to the thickness of the liner which is interposed between the insert and said interior surface of said cup.

40. An acetabular cup assembly according to claim 39 wherein the equatorial extension distance may be adjusted to accommodate liners of different thicknesses.

41. A method of construction of an acetabular cup assembly comprising the steps of;
a) forming an acetabular cup having a generally convex outer surface for engaging acetabular bone and a generally concave inner surface together defining a cup wall which has an apex and an equator; b) taking an insert capable of insertion inside the acetabular cup; the insert including a convex outer surface and a concave recess which receives a femoral head c) applying a wear inhibiting liner to the convex outer surface of the insert so that when the insert is inserted in the acetabular cup the liner is disposed between the generally concave inner surface of the cup and the convex outer surface of the insert;

42. A method according to claim 41 comprising the further step of increasing in length the circumferential wall of the cup a predetermined circumferential distance beyond the equator to accommodate the liner.

43. A method according to claim 42 comprising the step of fusing the insert to the liner so that the liner is integral with the insert.

44. A method according to claim 43 comprising the additional step of inserting the insert into the acetabular cup so that at least part of an outer wall of the liner engages at least part of the inner wall of the cup and the remainder of the outer wall of the liner defines a gap or space between the liner and the cup.

45. A method according to claim 44 wherein the metallic liner is titanium alloy or chrome cobalt and the insert is polyethylene.

46. A method according to claim 45 wherein the circumferential wall of the cup is extended between 2 mm-5 mm from the equator.

47. A method according to claim 46 wherein the outer surface of the metallic liner includes a straight wall region extending from near its equator in the direction of the apex and an arcuate portion in the region of its apex, the arcuate portion opposing the inner concave surface of the cup.

48. A method according to claim 47 wherein the cup has a substantially spherical outer surface.

49. A method according to claim 48 wherein the acetabular cup is formed so that an axial distance from the apex of the outer surface of the acetabular cup to its equator is less that the radial distance from the outer surface of the circumferential wall at the equator to its axis of symmetry.

50. A method according to claim 49 including the step of disposing the straight wall region of the liner at an angle within the range of 15-25 degrees from its axis of symmetry

51. A method according to claim 41 wherein the insert is made of polyethylene.

52. A method according to claim 51 wherein the liner is made of either chrome cobalt or titanium alloy.

53. An liner for attachment with an insert for an acetabular cup; the insert including a convex outer surface and a concave inner surface defining a body including a recess which receives a femoral head component; wherein the convex outer surface of the insert body receives thereon the liner which provides a wear inhibiting surface opposing at least part of an inner surface of the cup which receives and retains said insert.

54. A liner for an acetabular cup according to claim 53, wherein the liner is formed from a metallic material which opposes at least part of a concave inner surface of the cup.

55. A liner for an acetabular cup according to claim 54 wherein, the liner is integral with the insert.

56. A liner for an acetabular cup according to claim 55 wherein the insert body is polyethylene and the metallic liner is fused to said polyethylene.

57. A liner for an acetabular cup according to claim 56 wherein, the metallic liner is titanium alloy or chrome cobalt and when the insert is inserted by the cup, engages at least part of an inner surface of the acetabular cup.

58. A liner for an acetabular cup according to claim 57 wherein a circumferential wall of the liner and cup is increased in length a predetermined circumferential distance beyond an equator of the cup.

59. A liner for an acetabular cup according to claim 58 wherein the thickness of the liner at its apex influences the circumferential distance that the wall of the cup is extended from the equator.
